(19) United States
(12) Patent Application Publication
(10) Pub. No.: US 2015/0313564 A1
(43) Pub. Date: Nov. 5, 2015

(54) X-RAY DIAGNOSTIC APPARATUS

(71) Applicants: Kabushiki Kaisha Toshiba, Minato-ku (JP); Toshiba Medical Systems Corporation, Otawara-shi (JP)

(72) Inventor: Yusuke NARABU, Nasushiobara (JP)

(73) Assignees: Kabushiki Kaisha Toshiba, Minato-ku (JP); Toshiba Medical Systems Corporation, Otawara-shi (JP)

(21) Appl. No.: 14/797,729

(22) Filed: Jul. 13, 2015

Related U.S. Application Data
(63) Continuation of application No. PCT/JP2014/052899, filed on Feb. 7, 2014.

(30) Foreign Application Priority Data
Feb. 8, 2013 (JP) 2013-023583

(51) Int. Cl. A61B 6/00

(52) U.S. Cl. CPC A61B 6/487; A61B 6/548; A61B 6/4441; A61B 6/466

(57) ABSTRACT

According to one embodiment, an X-ray diagnostic apparatus includes an X-ray generator, an X-ray detector, a supporting body, and a supporting body moving structure. The X-ray generator generates X-rays to be irradiated on an object. The X-ray detector detects the X-rays. The supporting body supports the X-ray generator and the X-ray detector. The supporting body moving structure is provided on the supporting body and includes a rotator. The supporting body moving structure reciprocates the supporting body by rotational motion of the rotator in one direction.

### CROSS-REFERENCE TO RELATED APPLICATIONS

This application is a Continuation Application of No. PCT/JP2014/052899, filed on Feb. 7, 2014, and the PCT application is based upon and claims the benefit of priority from Japanese Patent Application No. 2013-023583, filed on Feb. 8, 2013, the entire contents of which are incorporated herein by reference.

### FIELD

Embodiments of the present disclosure relate generally to an X-ray diagnostic apparatus which can perform fluoroscopic imaging of an object to acquire image data for binocular stereoscopic vision.

### BACKGROUND

An X-ray diagnostic apparatus has made rapid progress in association with development in computer technology, and it is indispensable for medical treatment as of today. Especially, an X-ray diagnostic apparatus in the field of circulatory organs making progress in association with development in catheter manipulation targets arteries and veins throughout the body including the cardiovascular system, and generally performs fluoroscopic imaging of vessel regions of an object, to which a contrast agent is administered, so as to generate and display image data of the vessel regions.

An X-ray diagnostic apparatus for diagnosis of an abdominal region and a circulatory organ region includes an imaging system composed of an X-ray tube of an X-ray generating unit, a plane detector of an X-ray detecting unit, etc., a supporting unit configured to support the imaging system such as a C-arm etc., a table for loading an object, and so on. In such an X-ray diagnostic apparatus, fluoroscopic imaging from the optimum direction for an object is achieved by moving the above table and supporting unit in a desired direction.

Meanwhile, in recent years, various types of binocular stereoscopic vision techniques, in which three-dimensional observation is performed by using image data acquired at mutually different two imaging positions or in mutually different two directions, have come into practical use. A medical image diagnostic apparatus, which enables three-dimensional observation inside an object by applying the above-mentioned binocular stereoscopic vision techniques, has been considered. In such a medical image diagnostic apparatus, a device called a naked-eye stereoscopic display for performing binocular stereoscopic vision is known. The naked-eye stereoscopic display can give a parallax between both eyes of an operator of a medical image diagnostic apparatus (i.e. an observer of image data), without having to put special eye-glasses on the operator.

In addition, as binocular stereoscopic vision techniques, for example, techniques such as an active method and a passive method are known. For example, in the binocular stereoscopic vision technique of the active method, the first image data generated for the left eye and the second image data generated for the right eye are alternately displayed on a monitor of a display unit by switching between the first image data and the second image data at a predetermined cycle. Then, the operator observes the image data displayed on the display unit, via active shutter glasses etc. having a shutter function in synchronization with the above-mentioned (alternate) display cycle.

In the binocular stereoscopic vision technique of the passive method, polarized light of the first image data and polarized light of the second image data are controlled to become orthogonal to each other, and an operator observes the above image data via polarization glasses.

Because a lesion area, blood running, etc. inside an object can be three-dimensionally visualized by applying the above-mentioned conventional binocular stereoscopic vision techniques, accurate diagnosis and advanced medical treatment are expected.

In this case, the imaging system mounted on the supporting unit are alternately arranged at the first imaging position corresponding to the first image data and the second imaging position corresponding to the second image data, by reciprocating the supporting unit supporting the imaging system such as a C-arm etc. within a predetermined range. Then, three-dimensional observation of biological information can be achieved by displaying the first and second image data time-sequentially acquired at the respective imaging positions on the monitor of the display unit in parallel at a predetermined interval.

In order to obtain three-dimensional biological information with satisfactory real-time property by using the binocular stereoscopic vision technique, it is necessary to increase each frame number of the first and second image data time-sequentially acquired at the respective imaging positions, by performing X-ray fluoroscopic imaging while rapidly reciprocating the imaging system between the first and second imaging positions.

However, if the rapid reciprocal motion of the imaging system compatible with the binocular stereoscopic vision technique is performed by using the conventional moving structure which moves the supporting unit mounting the imaging system in a desired direction for the purpose of setting imaging positions and imaging directions, it is necessary to perform rapid reciprocal rotation of a rotation unit such as a motor etc. included in the moving structure so as to correspond to the rapid reciprocal motion of the imaging system. Therefore, there is a problem that accurate rotation control is extremely difficult, in addition to generation of large burden at the rotation unit etc.

### BRIEF DESCRIPTION OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing overall configuration of the X-ray diagnostic apparatus of the present embodiment;

FIG. 2 is a block diagram showing concrete configuration of the X-ray fluoroscopic imaging system included in the X-ray diagnostic apparatus of the present embodiment;

FIG. 3 is a schematic circuit diagram of a plane detector included in an X-ray detector of the X-ray diagnostic apparatus of the present embodiment;

FIG. 4 is a schematic oblique drawing showing concrete configuration of a supporting device and a bed included in the X-ray diagnostic apparatus of the present embodiment;

FIG. 5 is a block diagram showing various types of moving structures and rotating structures installed in the supporting device and the bed of the present embodiment, and a driving circuit configured to supply driving signals to these structures;

FIG. 6A and FIG. 6B are diagrams for explaining the first imaging position and the second imaging position of the stereoscopic vision imaging mode of the present embodiment;

FIG. 7A and FIG. 7B are diagrams showing configuration of the slide moving structure included in the supporting device of the X-ray diagnostic apparatus of the present embodiment;

FIG. 8A, FIG. 8B and FIG. 8C are diagrams for explaining the concrete operation of the slide moving structure included in the supporting device of the present embodiment;

FIG. 9A and FIG. 9B are diagrams for explaining the naked-eye binocular stereoscopic vision method using the first image data and the second image data acquired in the stereoscopic vision imaging mode of the present embodiment;

FIG. 10 is a flow chart showing the procedure of generating and displaying image data in the stereoscopic vision imaging mode of the present embodiment;

FIG. 11A and FIG. 11B are diagrams showing configuration of the slide moving structure in the modified version of the present embodiment; and

FIG. 12A and FIG. 12B are diagrams for explaining the concrete operation of the slide moving structure in the modified versions of the present embodiment.

### DETAILED DESCRIPTION

Hereinbelow, a description will be given of an X-ray diagnostic apparatus according to embodiments of the present invention with reference to the drawings.

In general, according to one embodiment, an X-ray diagnostic apparatus includes an X-ray generator, an X-ray detector, a supporting body, and a supporting body moving structure. The X-ray generator generates X-rays to be irradiated on an object. The X-ray detector detects the X-rays. The supporting body supports the X-ray generator and the X-ray detector. The supporting body moving structure is provided on the supporting body and includes a rotator. The supporting body moving structure reciprocates the supporting body by rotational motion of the rotator in one direction.

### Embodiments

The X-ray diagnostic apparatus of the present embodiment performs (a) X-ray fluoroscopic imaging of a reciprocal motion mode in which X-ray imaging is performed at a plurality of positions by reciprocating an X-ray imaging system and (b) X-ray fluoroscopic imaging of a normal mode (standard imaging mode) which aims to specify imaging positions appropriate for the above-mentioned plurality of positions etc. The X-ray diagnostic apparatus includes a slide moving structure (supporting body moving structure) for the normal mode which moves an X-ray generator and an X-ray detector (imaging system) mounted near both end parts of a C-arm in a desired direction at normal speed and a slide moving structure (supporting body moving structure) for the reciprocal motion mode which has a function of converting rapid rotational motion in one predetermined direction into rapid reciprocal motion and performs X-ray imaging at a plurality of predetermined positions by rapidly reciprocating the above imaging system within a predetermined range.

(Configuration and Functions of the Apparatus)

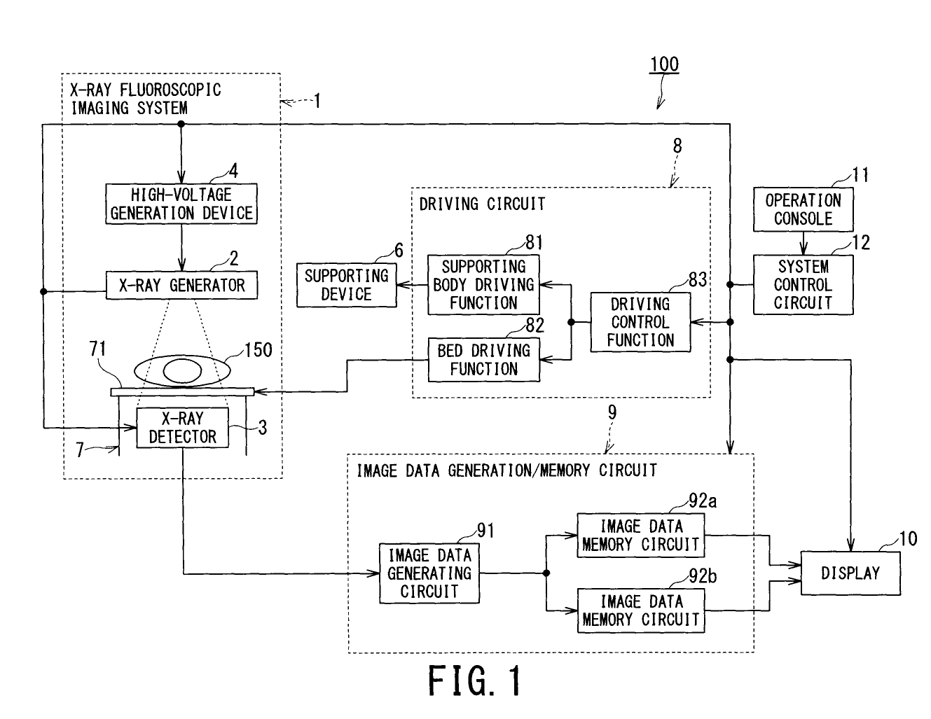

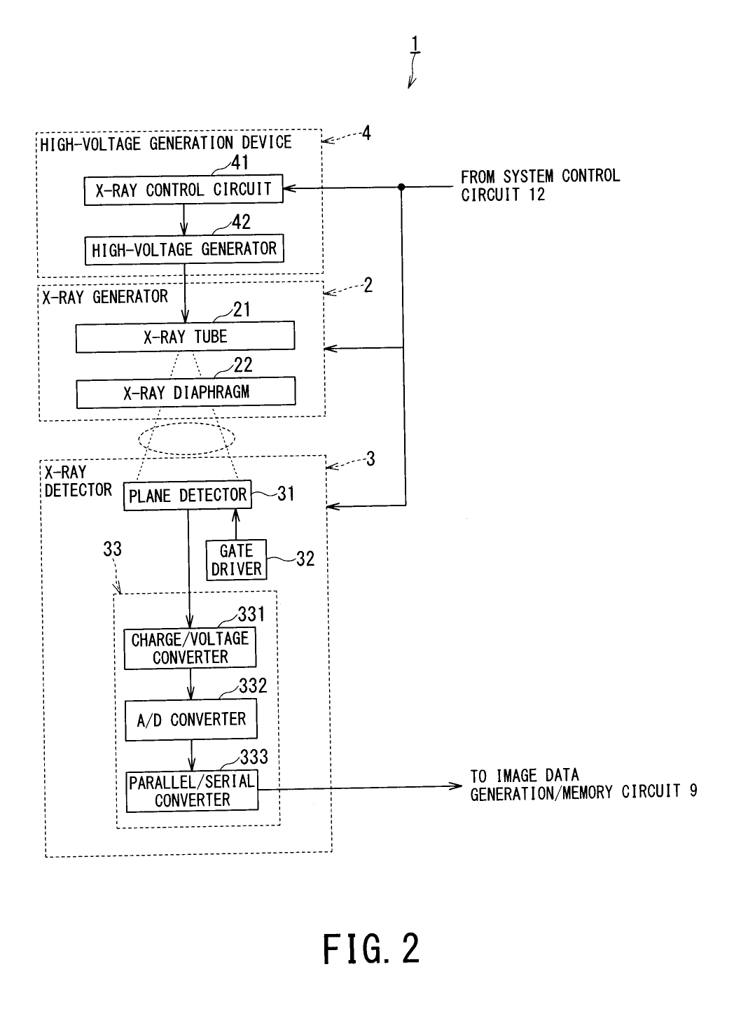

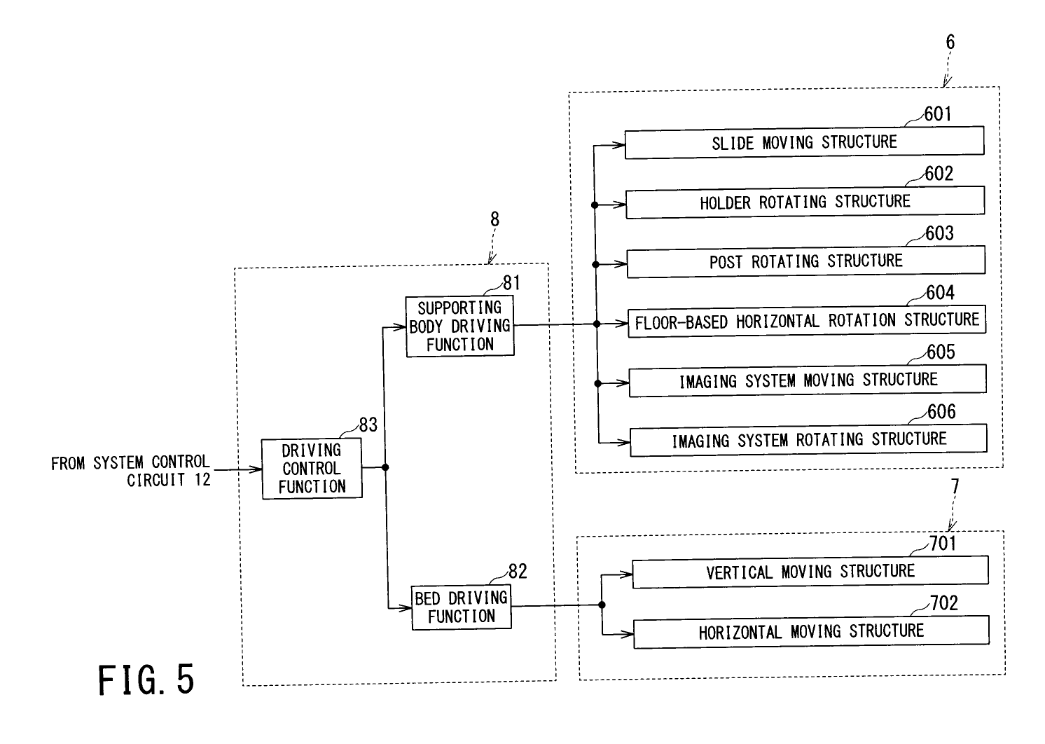

The configuration and functions of the X-ray diagnostic apparatus of the present embodiment will be explained with reference to FIG. 1 to FIG. 9 below. Incidentally, FIG. 1 is a block diagram showing the overall configuration of the X-ray diagnostic apparatus of the present embodiment, and FIG. 2 and FIG. 5 are block diagrams showing the concrete configurations of an X-ray fluoroscopic imaging system, a supporting body, and a driving circuit included in the above X-ray diagnostic apparatus.

As shown in FIG. 1, the X-ray diagnostic apparatus 100 of the present embodiment includes an X-ray fluoroscopic imaging system 1, a supporting device 6, and a table 71. The X-ray fluoroscopic imaging system generates projection data by performing X-ray fluoroscopic imaging of an object 150. The supporting device 6 supports an X-ray generator 2 and an X-ray detector 3 (to be described below) included in the X-ray fluoroscopic imaging system 1, and moves or rotates the X-ray generator 2 and the X-ray detector 3 around the object 150. The table 71 is disposed on the bed 7 and moves the object 150 loaded on its top surface to a position appropriate for X-ray fluoroscopic imaging.

In addition, the X-ray diagnostic apparatus 100 includes a driving circuit B. The driving circuit 8 supplies driving signals to various types of moving structures and rotating structures installed in the supporting device 6 and the bed 7. Thereby, the driving circuit 8 sets respective imaging positions suitable for X-ray fluoroscopic imaging of the normal mode (hereinafter, referred to as the standard imaging mode) and X-ray fluoroscopic imaging of the reciprocal motion mode, by moving the object 150 loaded on the table 71 and the imaging system mounted on the supporting body of the supporting device 6.

The normal mode (standard imaging mode) is a mode for moving the supporting device 6, along a predetermined direction. In the standard imaging mode, a user can perform positioning by moving the supporting device 6 to an arbitrary position and X-ray imaging at the determined position.

The reciprocal motion mode is a mode for reciprocating the supporting device 6 along a predetermined direction within a predetermined range by using rotational motion in one direction of a rotator. In the reciprocal motion mode, the supporting device 6 is made to reciprocate, and imaging at a plurality of predetermined (imaging) positions can be performed in one reciprocating period. Therefore, in the reciprocal motion mode, X-ray imaging can be repeatedly performed at a plurality of predetermined positions.

Thus, for example, if two positions including an imaging position for a left eye image and another imaging position for a right eye image suitable for generating a binocular stereoscopic vision image are defined as the plurality of predetermined positions, a left eye image and a right eye image for binocular stereoscopic vision can be easily acquired. In the present embodiment, an example in which imaging at two positions including an imaging position for a left eye image and another imaging position for a right eye image suitable for generating a binocular stereoscopic vision image is repeated in the reciprocal motion mode will be explained. In addition, in the explanation below, the reciprocal motion mode is referred to as the stereoscopic vision imaging mode.

In addition, the X-ray diagnostic apparatus 100 further includes an image data generation/memory circuit 9, a display 10, an operation console 11, and a system control circuit 12.

The image data generation/memory circuit 9 generates reference image data on the basis of projection data of the standard imaging mode generated by the X-ray fluoroscopic imaging system 1, and performs generation and storage of the first and second image data compatible with binocular stereoscopic vision on the basis of the projection data of the stereoscopic vision imaging mode.

The display 10 displays a stereoscopically visible image based on the plurality of X-ray images. The display 10 may display the reference image data obtained in the standard imaging mode and may display the first and second image data obtained in the stereoscopic vision imaging mode.

The operation console 11 performs selection of the imaging mode, setting of the reference imaging position in the standard imaging mode, setting of an imaging interval in the stereoscopic vision imaging mode, setting of fluoroscopic imaging conditions, input of various types of command signals, and so on.

The system control circuit 12 generally controls each of the above-mentioned components.

Hereinafter, the above-mentioned components of the X-ray diagnostic apparatus 100 will be explained more concretely.

The X-ray fluoroscopic imaging system 1 of the X-ray diagnostic apparatus 100 shown in FIG. 2 includes (a) the X-ray generator 2 which irradiates X-rays on the object 150, (b) the X-ray detector 3 which two-dimensionally detects X-rays having passed through the object 150 and generates the projection data on the basis of this detection result, and (c) a high-voltage generation device 4 which generates high voltage required for the above X-ray irradiation and supplies the X-ray generator 2 with the generated high voltage.

The X-ray generator 2 includes an X-ray tube 21 which radiates X-rays onto the object 150, and an X-ray diaphragm 22 which forms incident X-ray beam flux (cone beam) from the X-rays radiated from the X-ray tube 21.

The X-ray tube 21 is a vacuum tube generating X-rays, and generates X-rays by accelerating electrons emitted from its cathode (filament) under the high-voltage so as to collide against its tungsten anode.

The X-ray diaphragm 22 is used for the purpose of reducing exposure dose of the object 150 and improving image quality. This X-ray diaphragm 22 includes non-illustrated diaphragm blades and a non-illustrated compensation filter. The diaphragm blades adjust (set) a fluoroscopic imaging region of the object 150 which X-rays radiated from the X-ray tube 21 passes through. The compensation filter prevents halation by selectively reducing X-rays having passed through a living tissue whose X-ray absorption rate is low.

Meanwhile, the X-ray detector 3 includes a plane detector 31, a gate driver 32, and a projection data generation circuit 33. The plane detector 31 converts X-rays having passed through the fluoroscopic imaging region formed by the diaphragm blades of the X-ray diaphragm 22 into signal charge and accumulates the signal charge. The gate driver 32 reads out the signal charge accumulated in the plane detector 31. The projection data generation circuit 33 generates the projection data on the basis of the signal charge which is read out by the gate driver 32.

Incidentally, in X-ray detecting techniques, a method of directly converting X-rays into signal charge and a method of once converting X-rays into light and then converting the light into signal charge are included. Although the former is explained in the present embodiment as an example, the latter is also applicable to the present embodiment. In addition, a method of using an X-ray I.I. (Image Intensifier) instead of the plane detector 31 is also applicable to the present embodiment.

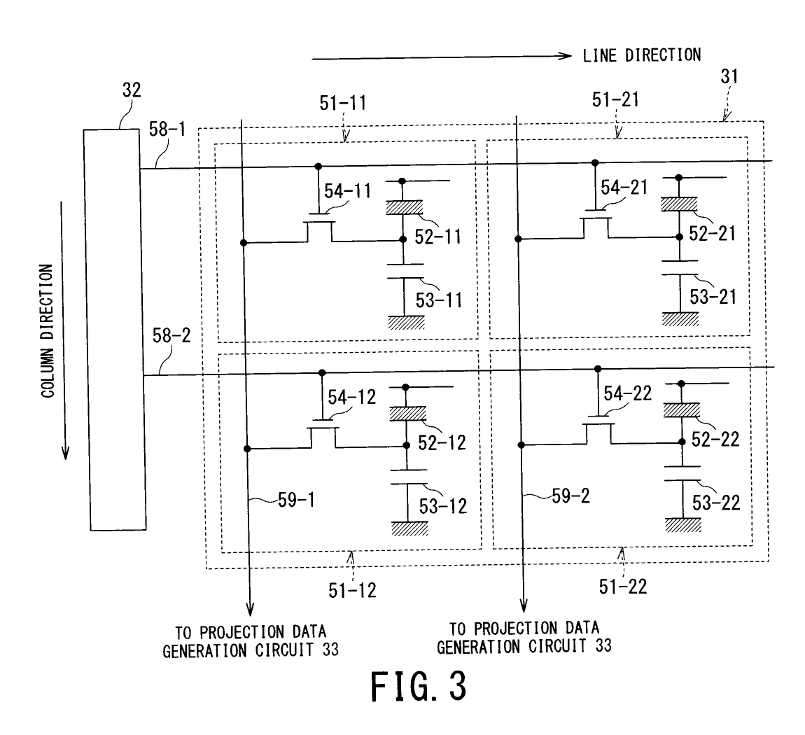

The plane detector 31 of the X-ray detector 3 is configured by two-dimensionally arranging tiny detection elements 51 in a column direction and a line direction as shown in FIG. 3. Each of the detection elements 51 (51-11, 51-12, 51-21, and 51-22) includes a photoelectric film 52 (52-11, 52-12, 52-21, and 52-22), a charge accumulation capacitor 53 (53-11, 53-12, 53-21, and 53-22), and a TFT 54 (Thin-Film Transistors 54-11, 54-12, 54-21, and 54-22). Each of the photoelectric films 52 detects X-rays and generates signal charge in accordance with incident X-ray amount. Each of the charge accumulation capacitors 53 accumulates the signal charge generated in the corresponding photoelectric film 52. Each of the TFTs 54 reads out the signal charge accumulated in the corresponding charge accumulation capacitor 53 at predetermined timing.

Incidentally, in order to simplifying the explanation, the plane detector 31 configured by arranging two detection elements 51 in the column direction (up-and-down direction in FIG. 3) and two detection elements 51 in the line direction (right-and-left direction in FIG. 3) like a two-dimensional matrix is explained in FIG. 3. However, the plane detector 31 used for actual X-ray fluoroscopic imaging is configured by arranging much more detection elements 51 in the column direction and in the line direction.

Meanwhile, the gate driver 32 inputs a driving pulse to each TFT 54 via the signal lines 58 (58-1 and 58-2), in order to read out the signal charge generated in the photoelectric film 52 of each detection element 51 and accumulated in the charge accumulation capacitor 53 of each detection element 51.

As shown in FIG. 2, the projection data generation circuit 33 includes a charge/voltage converter 331 configured to convert the signal charge being read out from the plane detector 31 into voltage, an A/D (analogue/digital) converter 332 configured to convert the output of charge/voltage converter 331 into digital signals, and a parallel/serial converter 333 configured to convert data elements of the projection data being read out per line in parallel from the plane detector 31 into time-series signals. In this case, the channel number of the charge/voltage converter 331 and the A/D converter 332 is the same as the channel number of the signal output lines 59 (59-1 and 59-2) of the plane detector 31 shown in FIG. 3.

The high-voltage generation device 4 of the X-ray fluoroscopic imaging system 1 includes a high-voltage generator 42 and an X-ray control circuit 41. The high-voltage generator 42 applies high voltage between the anode and the cathode in order to accelerate thermoelectrons generated from the cathode of the X-ray tube 21 installed in the X-ray generator 2. The X-ray control circuit 41 sets a tube current and a tube voltage of the X-ray tube 21, X-ray irradiation time, timing of irradiating X-rays, cycle of repeating irradiation, and so on, by controlling application voltage, application time, application timing, etc. of the high-voltage generator 42 on the basis of the X-ray irradiation conditions of the fluoroscopic imaging conditions inputted from the system control circuit 12.

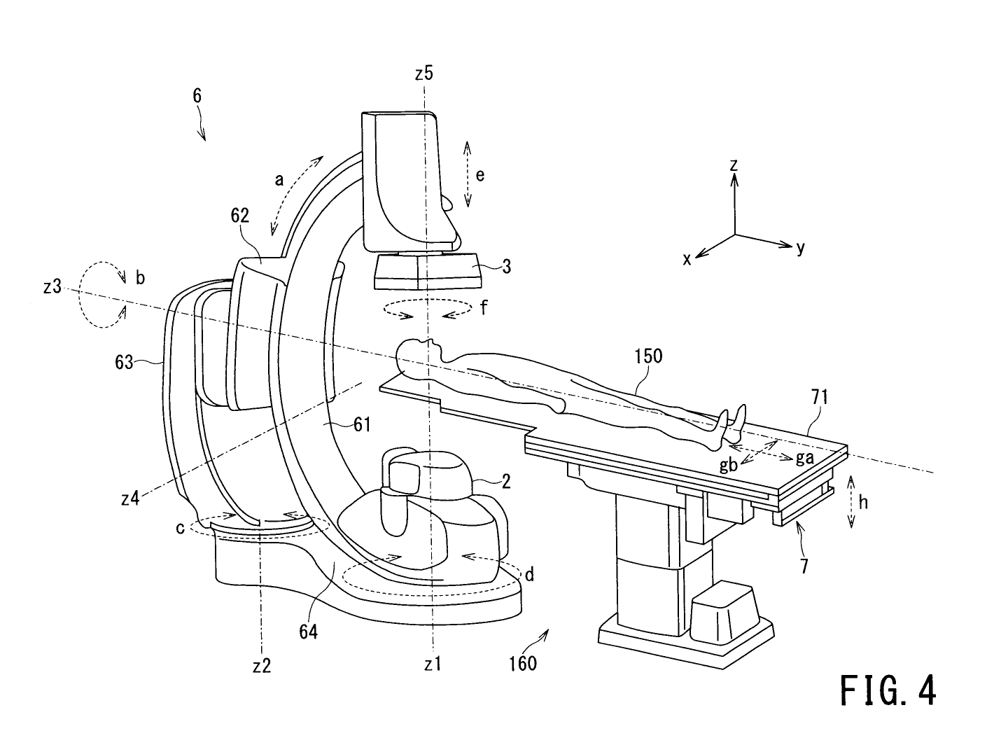

Next, concrete configurations and functions of the supporting device 6 and the bed 7 including the table 71 shown in FIG. 1 will be explained with reference to FIG. 4 and FIG. 5.

FIG. 4 shows the supporting device 6 including a C-arm (supporting member) 61 and the like, on both ends of the C-arm 61 the X-ray generator 2 and the X-ray detector 3 (imaging system) being mounted, and the bed 7 including the table 71 on which the object 150 is loaded. Incidentally, in the explanation below, the body axis direction of the object 150 (longitudinal direction of the table 71) is defined as a y-direction, a direction orthogonal to a floor plane 160 on which the supporting device 6 and the bed 7 are installed is defined as a z-direction, and the direction orthogonal to both y-direction and z-direction (i.e. the width direction of the table 70) is defined as an x-direction, as shown in FIG. 4.

In addition, FIG. 5 is a block diagram showing an example of configuration of (a) various types of moving structures and rotating structures installed in the supporting device 6 and the bed 7 and (b) the driving circuit 8 which supplies driving signals to these structures.

The supporting device 6 includes a supporting body including the C-arm 61, an arm holder 62, an arm supporting post 63, and a floor-based horizontal rotation arm 64. The one end part of the floor-based horizontal rotation arm 64 is rotatably installed, so that it can freely rotate about a floor rotation axis z1 orthogonal to the floor plane 160 in the direction of the arrow d. In addition, the arm supporting post 63 is rotatably mounted on the opposite end of the floor-based horizontal rotation arm 64, so as to be rotatable in the direction of the arrow c around an aim supporting post rotational axis z2 which is in parallel with the z-direction.

Moreover, the arm holder 62 is mounted on the lateral surface of the arm supporting post 63, so that it can rotate in the direction of the arrow b around an arm main rotational axis z3 which is in parallel with the y-direction. The C-arm 61 is mounted on the lateral surface of the arm holder 62 so as to be freely slidable in the direction of the arrow a around the arm slide central axis z4, and the X-ray generator 2 and the X-ray detector 3 are mounted near both end parts of the C-arm so as to face each other.

In addition, the X-ray detector 3 of the imaging system mounted near one end of the C-arm 61 can move in the direction of the arrow e. In addition, this X-ray detector 3 is installed, so that it can freely rotate about the imaging system rotation axis z5 in the direction of the arrow f in conjunction with the X-ray diaphragm 22 installed in the X-ray generator 2.

Furthermore, as shown in FIG. 5, each of the above-mentioned components of the supporting device 6 includes a slide moving structure (supporting body moving structure) 601, a holder rotating structure 602, a post rotating structure 603, a floor-based horizontal rotation structure 604, an imaging system moving structure 605, and an imaging system rotating structure 606.

The slide moving structure (supporting body moving structure) 601 slides the C-arm 61 in the a-direction in FIG. 4 about the arm slide central axis z4 as the center. The holder rotating structure 602 rotates the arm holder 62 about the arm main rotational axis z3 in the b-direction in FIG. 4. The post rotating structure 603 rotates the arm supporting post 63 about the arm supporting post rotational axis z2 in the c-direction in FIG. 4. The floor-based horizontal rotation structure 604 rotates the floor-based horizontal rotation arm 64 about the floor-based rotational axis z1 in the d-direction in FIG. 4. The imaging system moving structure 605 moves the X-ray detector 3 in the e-direction in FIG. 4. The imaging system rotating structure 606 rotates the X-ray detector 3 about the imaging system rotational axis z5 in the f-direction in FIG. 4.

Meanwhile, the bed 7 includes a vertical moving structure 701 and a horizontal moving structure 702. The vertical moving structure 701 upwardly and downwardly moves the table 71, on which the object 150 is loaded, in the h-direction (z-direction) in FIG. 4. The horizontal moving structure 702 slides the table 71 in the longitudinal direction ga (y-direction) or in the width direction gb (x-direction) in FIG. 4.

Accordingly, the imaging system mounted near both end parts of the C-arm 61 can be located to a position suitable for X-ray fluoroscopic imaging of the object 150 loaded on the table 71, by driving the above-mentioned moving structures and rotating structures so as to move the respective components installed in the supporting device 6 and the bed 7 in a desired direction.

Especially, when the stereoscopic imaging mode is selected as an imaging mode, two imaging positions (i.e. the first and second imaging positions) suitable for binocular stereoscopic vision can be determined by driving the above-mentioned slide moving structure (supporting body moving structure) 601 so as to slide the C-arm 61, on which the imaging system is mounted, within a predetermined angle in a reciprocating manner. As to a concrete moving method, it will be described below.

The driving circuit 8 includes at least a processor and a memory circuit. The driving circuit 8 conducts at least a supporting body driving function 81, a bed driving function 82, and a driving control function 83 by making the processor execute respective programs stored in the memory circuit (see FIG. 1). The driving control function 83 is a function of generating a driving control signal which controls the supporting body driving function 81 and the bed driving function 82 so as to control the driving of the supporting device 6 and the bed 7, on the basis of a control signal inputted from the system control circuit 12. The supporting body driving function 81 includes a function of generating a driving signal, which moves the imaging system around the object 150 (hereinafter, the driving signal for driving the supporting body will be referred to as the first driving signal), on the basis of the driving control signal generated by the driving control function 83, a function of supplying the first driving signal to the moving structures, and a function of rotating structures installed in the supporting device 6. The bed driving function 82 includes a function of generating a driving signal, which moves the table 71 with the object 150 loaded thereon (hereinafter, the driving signal for driving the table 71 will be referred to as the second driving signal), on the basis of the driving control signal generated by the driving control function 83, and a function of supplying the second driving signal to the moving structures installed in the bed 7.

Then, the driving circuit 8 in the standard imaging mode arranges the imaging system mounted near both end parts of the C-arm 61 at the reference imaging position suitable for acquiring the reference image data, by supplying the first and second driving signals to the various types of the moving structures and rotating structures installed in the supporting device 6 and the bed 7 so as to move the C-arm 61 and the table 71.

By the supporting body driving function 81, the driving circuit 8 in the stereoscopic vision imaging mode arranges the imaging system mounted near both end parts of the C-arm 61 at the first imaging position and the second imaging position suitable for binocular stereoscopic vision, by driving the slide moving structure 601 of the supporting device 6 so as to rapidly perform reciprocal sliding motion of the C-arm 61 within a predetermined angle.

Incidentally, the first imaging position where the first image data are acquired and the second imaging position where the second image data are acquired are generally determined/set by using the reference imaging position determined in the standard imaging mode as the center.

FIG. 5 shows the configurations of the various types of the moving structures and rotating structures installed in the supporting device 6 and the bed 7 and the driving circuit 8 supplying the driving signals to these structures. The slide moving structure 601 configured to slide the C-arm 61 along the (arc) running direction is installed in the joint part between the C-arm 61 and the arm holder 62 of the supporting device 6 shown in FIG. 4. In addition, the holder rotating structure 602 configured to rotate the arm holder 62 in the b-direction is installed in the joint part between the arm holder 62 and the arm supporting post 63. Moreover, the post rotating structure 603 configured to rotate the arm supporting post 63 in the c-direction is installed in the joint part between the arm supporting post 63 and the floor-based horizontal rotation arm 64. In addition, the floor-based horizontal rotation structure 604 configured to rotate the floor-based horizontal rotation arm 64 in the d-direction is installed in the joint part between the floor-based horizontal rotation arm 64 and the floor plane 160. In addition, the imaging system moving structure 605 configured to move the imaging system in the e-direction and the imaging system rotating structure 606 configured to rotate the imaging system in the f-direction are installed in the joint part between the imaging system and the end part of the C-arm 61.

The slide moving structure 601, the holder rotating structure 602, the post rotating structure 603, the floor-based horizontal rotation structure 604, the imaging system moving structure 605, and the imaging system rotating structure 606 each include structures of controlling movement and rotation such as a motor, a gear, a belt, a pulley, etc.

Meanwhile, the vertical moving structure 701 configured to upwardly and downwardly move the table 71 with the object 150 loaded thereon in the h-direction and the horizontal moving structure 702 configured to slide the table 71 in the longitudinal direction (direction ga) or in the width direction (direction gb) are installed in the bed 7.

Then, the first driving signal from the supporting body driving function 81 of the driving circuit 8 is supplied to each of the slide moving structure 601, the holder rotating structure 602, the post rotating structure 603, the floor-based horizontal rotation structure 604, the imaging system moving structure 605, and the imaging system rotating structure 606 of the supporting device 6. In addition, the second driving signal from the bed driving function 82 of the driving circuit 8 is supplied to each of the vertical moving structure 701 and the horizontal moving structure 702 of the bed 7.

In other words, the driving circuit 8 sets the reference imaging position of the standard imaging mode and the first and second imaging positions of the stereoscopic vision imaging mode, by supplying the above-mentioned first and second driving signals to the various types of moving structures and rotating structures installed in the supporting device 6 and the bed 7 so as to move the imaging system installed near both end parts of the C-arm 61 and the table 71 with the object 150 loaded thereon.

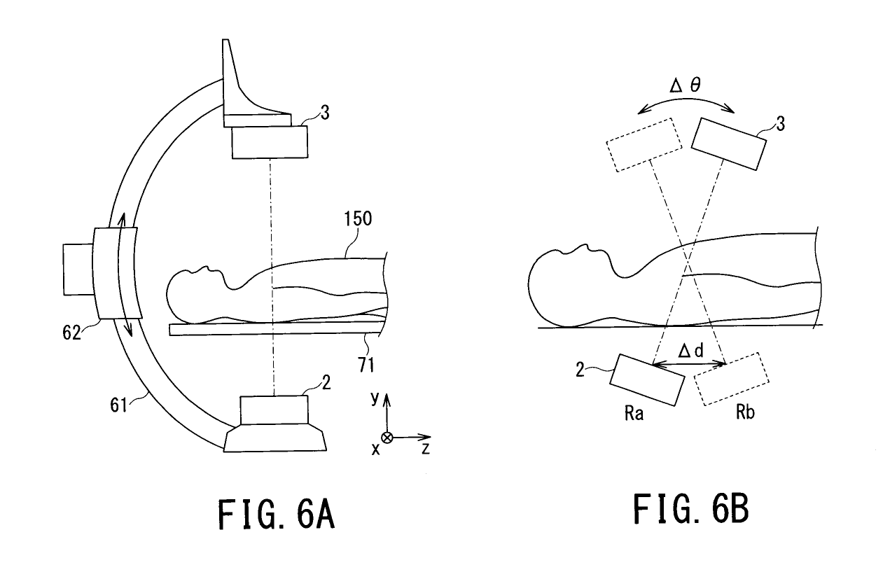

Next, the reciprocal slide motion of the C-arm 61 achieved by the slide moving structure 601 of the supporting device 6 and the first and second imaging positions of the stereoscopic vision imaging mode set by this reciprocal slide motion will be explained with reference to FIG. 6A and FIG. 6B.

FIG. 6A shows the direction of the reciprocal slide motion of the C-arm 61 in the stereoscopic vision imaging mode (with the arc bidirectional arrow), and the X-ray detector 3 and the X-ray generator 2 are mounted near the upper end part and the lower end part of this C-arm 61. FIG. 6B shows the first imaging position Ra and the second imaging position Rb suitable for binocular stereoscopic vision to be set this time.

In other words, when the reciprocal slide motion of the C-arm 61 within the angle Δθ is rapidly performed by the slide moving structure 601 of the supporting device 6, the X-ray generator 2 and the X-ray detector 3 (imaging system) are made to rapidly move around the object 150 in a reciprocating manner together with the C-arm 61. At this time, for example, the first imaging position Ra and the second imaging position Rb are set to both turnaround points of the rapid reciprocal motion, which are mutually separated by a predetermined imaging interval Δd. Incidentally, though the first imaging position Ra and the second imaging position Rb are set to the positions of the X-ray generator 2 irradiating X-rays for fluoroscopy/imaging on the object 150 here, setting of the first imaging position Ra and the second imaging position Rb is not limited to this example.

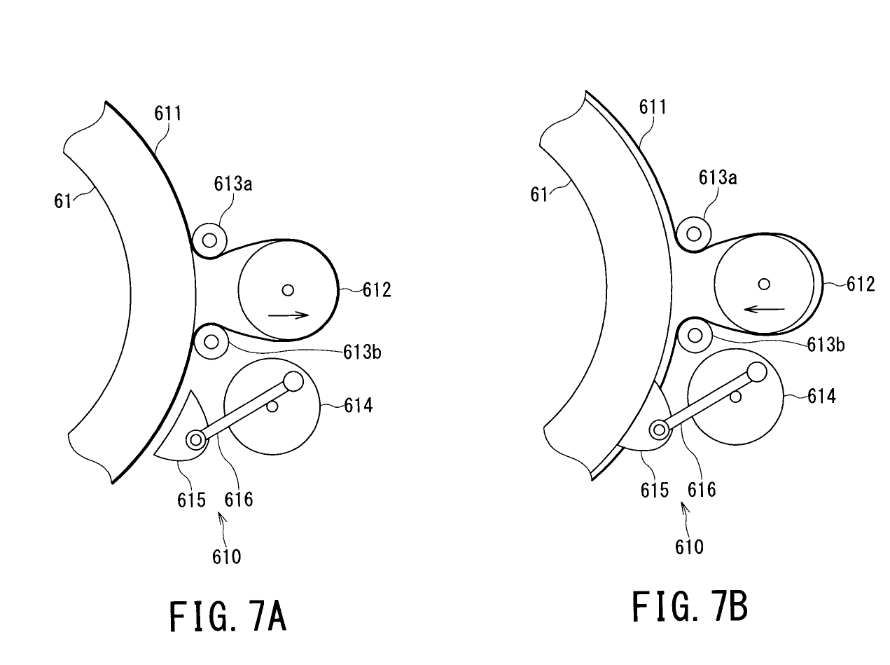

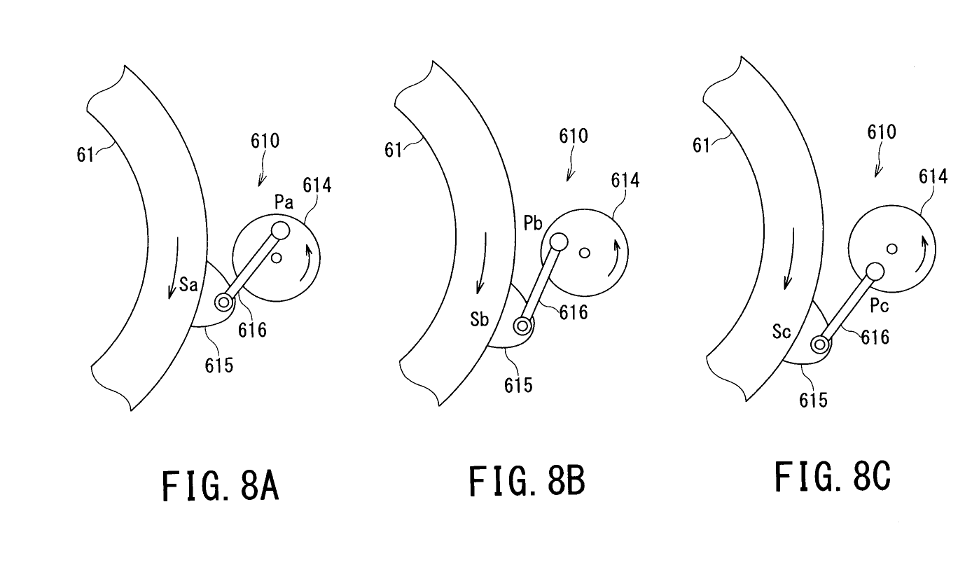

Next, the configuration and function of the slide moving structure 601 in the standard imaging mode and the stereoscopic vision imaging mode will be explained with reference to FIG. 7A and FIG. 7B, and the concrete operation of the slide moving structure 601 in the stereoscopic vision imaging mode will be explained with reference to FIG. 8A to FIG. 8C.

As shown in FIG. 7A, the slide moving structure 601 of the present embodiment includes a belt 611, a rotator 612, pulleys 613a and 613b, and a link structure 610.

The belt 611 is mounted along the lateral surface of the C-arm 61. The rotator 612 slides the C-arm 61 in a predetermined direction by moving the belt 611. The pulley 613a changes the running direction of the belt 611 from the lateral surface of the C-arm 61 into the lateral surface of the rotator 612. The pulley 613b changes the above-mentioned running direction from the lateral surface of the rotator 612 into the lateral surface of the C-arm 61. The link structure 610 includes a roller 614 as a rotator rapidly spinning at predetermined rate, a mounting fixture 615 detachable to the lateral surface of the C-arm 61 by a locking means such as an electromagnet etc., and an arm 616 whose one end is rotatably mounted near the peripheral part of the roller 614, and whose other end is rotatably mounted on the mounting fixture 615.

Then, when the standard imaging mode is selected by the operation console 11, the driving circuit 8 supplies the first driving signal by the supporting body driving function 81 to the slide moving structure 601 of the supporting device 6 so as to release the locked state between the mounting fixture 615 of the link structure 610 and the lateral surface of the C-arm 61 as shown in FIG. 7A.

Next, the supporting body driving function 81 of the driving circuit 8 rotates the rotator 612 in a predetermined direction under the state where the belt 611 mounted on the lateral surface of the C-arm 61 is tensed, by controlling the rotator 612 so as to move to the right. Thereby, the C-arm 61 linked with the rotator 612 via the belt 611 slides along its running direction in conjunction with the rotation of the rotator 612, and the imaging system (FIG. 4) mounted near both end parts of the C-arm 61 moves. As described above, in the standard imaging mode, a user can perform positioning by moving the supporting device 6 to an arbitrary position via the operation console 11 or manually.

Meanwhile, when the stereoscopic vision imaging mode is selected, the driving circuit 8 supplies the first driving signal generated in the same way as the standard imaging mode by the supporting body driving function 81 to the slide moving structure 601, and releases the linked state between the belt 611 and the C-arm 61 by moving the rotator 612 to the left so as to bring the belt 611 mounted on the lateral surface of the C-arm 61 into the loosened state as shown in FIG. 7B.

Next, the supporting body driving function 81 achieves the rapid reciprocal slide motion of the C-arm 61 within the predetermined angle Δθ, by mounting the mounting fixture 615 of the link structure 610 on the lateral surface of the C-arm 61 and then controlling the roller 614 so as to rapidly rotate. Thereby, the driving circuit 8 rapidly moves the imaging system mounted near both end parts of the C-arm 61 in a reciprocating manner between the first and second imaging positions suitable for the stereoscopic vision imaging mode.

Incidentally, though the rotational structures for rotating the rotator 612 and the roller 614 such as a motor etc. are installed inside the rotator 612 or the roller 614 in general, the rotational structures may be separately provided.

FIG. 8A to FIG. 8C show the state wherein the mounting fixture 615 mounted on the lateral surface of the C-arm 61 rapidly reciprocates in conjunction with the rapid spin of the roller 614 in one direction in the stereoscopic imaging mode. For example, when the joint part between the arm 616 and the roller 614 rapidly rotating counterclockwise moves to the position Pa in FIG. 8A, the mounting fixture 615 moves to the position Sa corresponding to the first imaging position along the running direction of the C-arm 61. When the above-mentioned joint part moves to the position Pc in FIG. 8C, the mounting fixture 615 moves to the position Sc corresponding to the second imaging position.

In other words, the mounting fixture 615 of the link structure 610 mounted on the lateral surface of the C-arm 61 repeats the rapid reciprocal motion between the position Sa and the position Sc in association with the rapid rotation of the roller 614 in one direction, and the C-arm 61 rapidly performs a reciprocal slide motion along its running direction caused by the rapid reciprocal motion of the mounting fixture 615. Then, when the mounting fixture 615 mounted on the C-arm 61 reaches the position Sa, the imaging system mounted near both end parts of the C-arm 61 is arranged at the first imaging position suitable for the stereoscopic imaging mode. Similarly, when the mounting fixture 615 reaches the position Sc, the above-mentioned imaging system is arranged at the second imaging position.

Incidentally, in the above explanation, an example, in which both switching points (turnaround points) Sa and Sc of the movement direction in the reciprocal motion are treated as the first and second imaging positions respectively, has been shown. However, the first and second imaging positions are not limited to the switching points of a reciprocal motion, but they may be arranged within a predetermined range where the reciprocal motion is performed.

As shown in FIG. 1, the image data generation/memory circuit 9 includes an image data generating circuit 91 and image data memory circuits 92a and 92b. The image data generation/memory circuit 9 also includes a projection data memory circuit not shown.

The image data generating circuit 91 includes at least a processor and a memory circuit. The image data generating circuit 91 conducts at least, by making the processor execute programs stored in the memory circuit, a function of generating two-dimensional image data by sequentially storing data elements of the projection data, which are time-sequentially supplied from the projection data generation circuit 33 included in the X-ray detector 3 of the X-ray fluoroscopic imaging system 1, in the above-mentioned projection data memory circuit. Especially, in the stereoscopic imaging mode, this function of the image data generating circuit 91 generates the first image data based on the projection data supplied from the projection data generation circuit 33 during implementation term of X-ray fluoroscopic imaging at the first imaging position, and generates the second image data based on the projection data supplied from the projection data generation circuit 33 during implementation term of X-ray fluoroscopic imaging at the second imaging position.

The image data generating circuit 91 stores the first image data in the image data memory circuit 92a, stores the second image data in the image data memory circuit 92b.

The display 10 has a naked-eye stereoscopic display function of giving a binocular parallax without performing parallel display and without having to put special eyeglasses on a medical worker (hereinafter, referred to as an operator) who operates the X-ray diagnostic apparatus 100, or has a function of displaying the first and second image data being read out from the image data memory circuit 92a and 92b, respectively, in parallel on its own monitor.

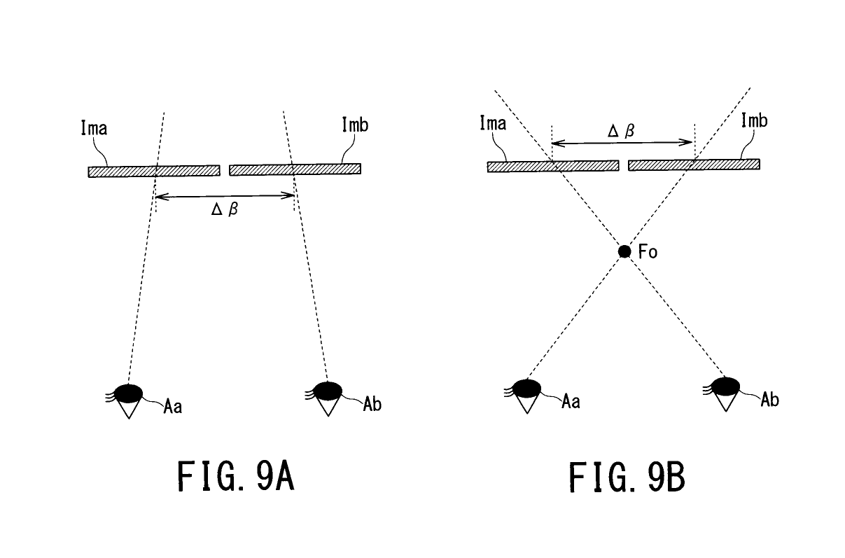

In the following explanation, when it is described as “a naked-eye stereoscopic display as the display 10”, it means a monitor which gives a binocular parallax to an operator without any need to perform parallel display. In addition, when it is simply described as “the display 10”, it means a monitor capable of displaying a right eye image and a left eye image in parallel as shown in FIG. 9A and FIG. 9B.

Incidentally, a mechanism of making mutually different light beams separately incident on the right eye and the left eye by using various types of techniques such as a parallax barrier type, a lenticular lens type, etc. is installed in a naked-eye stereoscopic display having a function of giving a binocular parallax without having to put special eyeglasses on an operator. However, these kinds of techniques are well known and their explanation is omitted.

The display 10 includes a non-illustrated monitor, and includes at least a processor and a memory circuit. The display 10 conducts at least a display data generation function and a conversion processing function by making the processor execute respective programs stored in the memory circuit. The display data generation function generates display data by, for example, arranging the first and second image data being read out from the image data memory circuit 92a and 92b, respectively, in parallel at intervals suitable for naked-eye binocular stereoscopic vision. The conversion processing function performs conversion processing such as D/A (digital/analogue) conversion, television format conversion, etc. on these display data. The monitor displays the display data subjected to the conversion processing.

Incidentally, the data interval between the first image data and the second image data displayed on the display 10 is set based on the imaging interval Δd preliminarily determined by the operation console 11 to be described below.

The operation console 11 is an interactive interface which includes operation/input devices such as a display panel, a key board, a trackball, a joystick, a mouse, etc. The operation console 11 performs input of object information, selection of the imaging mode (standard imaging mode/stereoscopic vision imaging mode), setting of the fluoroscopic imaging conditions including the X-ray irradiation conditions (a tube current, a tube voltage, X-ray irradiation time, X-ray irradiation cycle, timing of irradiating X-rays, etc.), setting of conditions of generating image data, setting of the imaging interval Δd in the stereoscopic vision imaging mode, input of various types of command signals, and so on.

The system control circuit 12 includes at least a processor and a memory circuit and includes an input information memory circuit. Various types of information inputted/set/selected in the operation console 11 are stored in the input information memory circuit. The system control circuit 12 conducts at least a function of acquisition of the reference image data and a function of setting of the reference imaging position by making the processor execute respective programs stored in the memory circuit. These reference image data can be acquired, by generally controlling the above-mentioned respective components of the X-ray diagnostic apparatus 100 based on the above-mentioned information being read out from the input information memory circuit so as to make the X-ray diagnostic apparatus 100 perform X-ray fluoroscopic imaging of the standard imaging mode on a fluoroscopic imaging region of the object 150. Moreover, the CPU makes the X-ray diagnostic apparatus 100 perform generation and display of the first and second image data compatible with binocular stereoscopic vision, by making the imaging system rapidly reciprocate between the first and second imaging positions suitable for the stereoscopic vision imaging mode determined based on the reference imaging position as the center.

Next, the naked-eye binocular stereoscopic vision technique with the use of the first and second image data of the stereoscopic vision imaging mode will be explained with reference to FIG. 9A and FIG. 9B. In the naked-eye binocular stereoscopic vision technique, for example, the first image data generated for the left eye and the second image data generated for the right eye are displayed in parallel on the monitor of the display 10 at predetermined data intervals. In this case, an operator can directly observe these image data displayed on the monitor without using special polarization glasses.

In general, this binocular stereoscopic vision technique includes a parallel method shown in FIG. 9A and a crossing method shown in FIG. 95.

The parallel method is a method of arranging the first image data Ima and the second image data Imb displayed on the monitor of the display 10 in parallel at predetermined data interval Δβ in front of a non-illustrated focal point Fo of the left eye Aa and the right eye Ab. The crossing method is a method of arranging the first image data Ima and the second image data Imb farther than the focal point Fo of the left eye Aa and the right eye Ab. Either the parallel method or the crossing method can be applied to the naked-eye binocular stereoscopic vision of the present embodiment.

(Procedure of Generation and Display of Image Data)

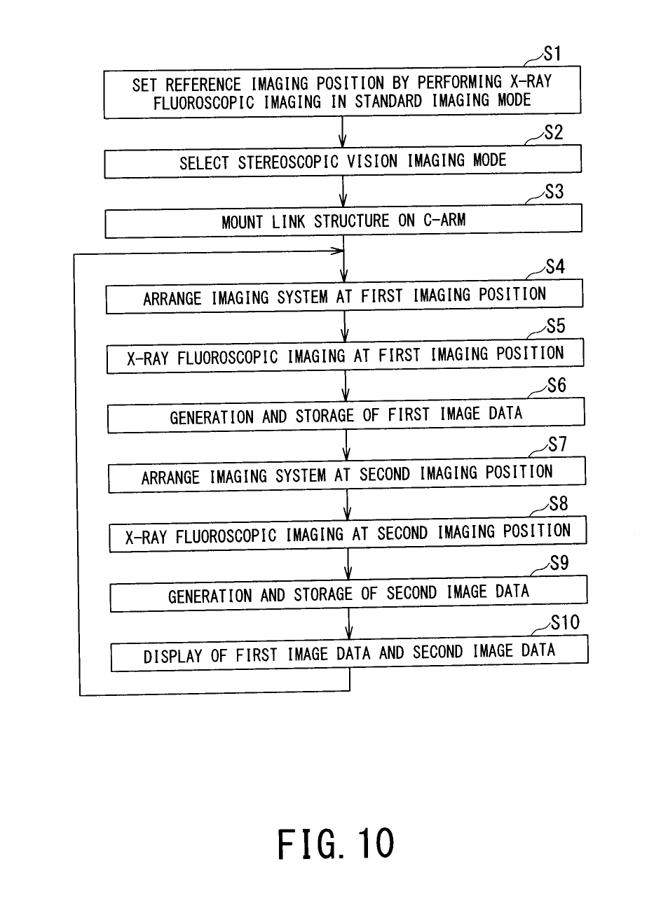

Next, the generation/display procedure of image data for the purpose of achieving binocular stereoscopic vision in the present embodiment will be explained with reference to the flow chart in FIG. 10.

Prior to acquisition of the first and second image data compatible with the binocular stereoscopic vision technique, the X-ray fluoroscopic imaging system 1 of the X-ray diagnostic apparatus 10 performs X-ray fluoroscopic imaging of the object 150 in the standard imaging mode, by using the imaging system moving in conjunction with the C-arm 61 according to a command signal to move the supporting body inputted from the operation console 11 via the system control circuit 12. Then, an operator performs acquisition of the reference image data and setting of the reference imaging position where these reference image data can be acquired, by adjusting the position of the imaging system under observation of the image data acquired this time (see the step S1 of FIG. 10).

Next, an operator selects the stereoscopic vision imaging mode by using an operation/input device of the operation console 11 (see the step S2 of FIG. 10). Afterward, the driving circuit B receives the above-mentioned selection information via the system control circuit 12, then generates the driving control signal for rapidly performing the reciprocal slide motion of the C-arm 61, and then supplies various types of first driving signals to the slide moving structure 601 of the supporting device 6. Thereby, the driving circuit 8 controls the belt 611 mounted on the lateral surface of the C-arm 61 to become the loosened state by moving the rotator 612 of the slide moving structure 601, and releases the locked state between the C-arm 61 and the rotator 612. Afterward, the slide moving structure 601 mounts the mounting fixture 615 of the link structure 610 on the lateral surface of the C-arm 61, in accordance with the above first driving signal (see the step S3 of FIG. 10).

Next, the slide moving structure 601 makes the C-arm 61 rapidly move along its running direction by rapidly rotating the roller 614 of the link structure 610 in a predetermined direction in accordance with the above first driving signal, and makes the imaging system mounted near both end parts of this C-arm 61 move from the reference imaging position of the standard imaging mode to the first imaging position suitable for the stereoscopic vision imaging mode (see the step S4 of FIG. 10).

Then, when the arrangement of the imaging system to the first imaging position is completed, the system control circuit 12 inputs a command signal to perform fluoroscopic imaging and X-ray irradiation conditions being read out from its own input information memory circuit into the X-ray control circuit 41 of the high-voltage generation device 4. The X-ray control circuit 41, which has received the command signal to perform fluoroscopic imaging, applies a predetermined high voltage to the X-ray tube 21 of the X-ray generator 2 by controlling the high-voltage generator 42 on the basis of the above-mentioned X-ray irradiation conditions. Then, the X-ray tube 21 subjected to application of the high voltage irradiates X-rays on the fluoroscopic imaging region of the object 150 via the X-ray diaphragm 22, and X-rays having passed through the fluoroscopic imaging region are detected by the plane detector 31 of the X-ray detector 3 arranged behind the object 150.

At this time, the photoelectric film 52 of each of the detection elements 51 two-dimensionally arranged in the plane detector 31 detects X-rays which have passed through the object 150, and accumulates signal charge in proportion to the transmission amount in the charge accumulation capacitor 53. Then, when irradiation of X-rays for a predetermined period is completed, the gate driver 32 sequentially reads out the signal charge accumulated in the charge accumulation capacitor 53 by supplying a driving pulse to each TFT 54 of the plane detector 31. Afterward, the signal charge being read out by the gate driver 32 is converted into voltage in the charge/voltage converter 331 of the projection data generation circuit 33, then the converted voltage is further converted into digital signals in the A/D converter 332, and then the digital signals are stored in a buffer memory of the parallel/serial converter 333 as projection data of one line (see the step S5 of FIG. 10).

Next, the parallel/serial converter 333 serially reads out data elements of the projection data stored in its own memory, and sequentially stores the data elements in the image data generation/memory circuit 9. Thereby, the first image data are generated in the image data generation/memory circuit 9. Then, the generated first image data are stored in the image data memory circuit 92a (see the step S6 of FIG. 10).

When the X-ray fluoroscopic imaging at the first imaging position and generation/storage of the first image data are completed, the slide moving structure 601 rapidly moves the C-arm 61 in the opposite direction by continuously performing rapid rotation of the roller 614 in accordance with the above first driving signal. Thereby, the slide moving structure 601 arranges the imaging system mounted near both end parts of this C-arm 61 to the second imaging position suitable for the stereoscopic vision imaging mode (see the step S7 of FIG. 10).

Then, when the arrangement of the imaging system to the second imaging position is completed, the X-ray fluoroscopic imaging at the second imaging position is performed in the way similar to the above-mentioned step S5 and the image data generation/memory circuit 9 generates the second image data on the basis of the projection data acquired by this X-ray fluoroscopic imaging. Afterward, the acquired second image data are stored in the image data memory circuit 92b (see the step S8 and the step S9 of FIG. 10).

When the generation and storage of the first and second image data are completed in the above manner, the display 10 performs display so as to give a binocular parallax to an operator based on the first and second image data, in the case of using a naked-eye stereoscopic display as a display means. When a naked-eye stereoscopic display is not used, the display 10 displays the first and second image data being read out from the image data memory circuit 92a and 92b, respectively, in parallel at data interval Δβ, which is determined based on the imaging interval Δd so as to become suitable for binocular stereoscopic vision (see the step S10 of FIG. 10).

When the display of the first and second image data acquired by the first rapid reciprocal motion of the imaging system is completed, the time-sequential first and second image data with high time resolution are displayed on the display 10 in parallel by repeating the above-mentioned processings of the step S4 to the step S10.

### Modified Embodiments

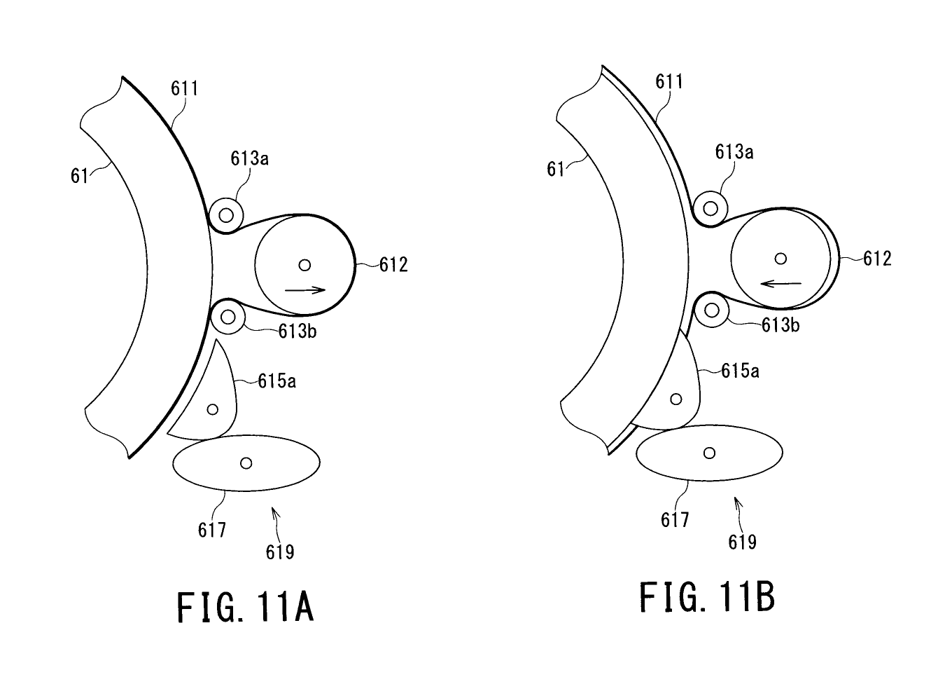

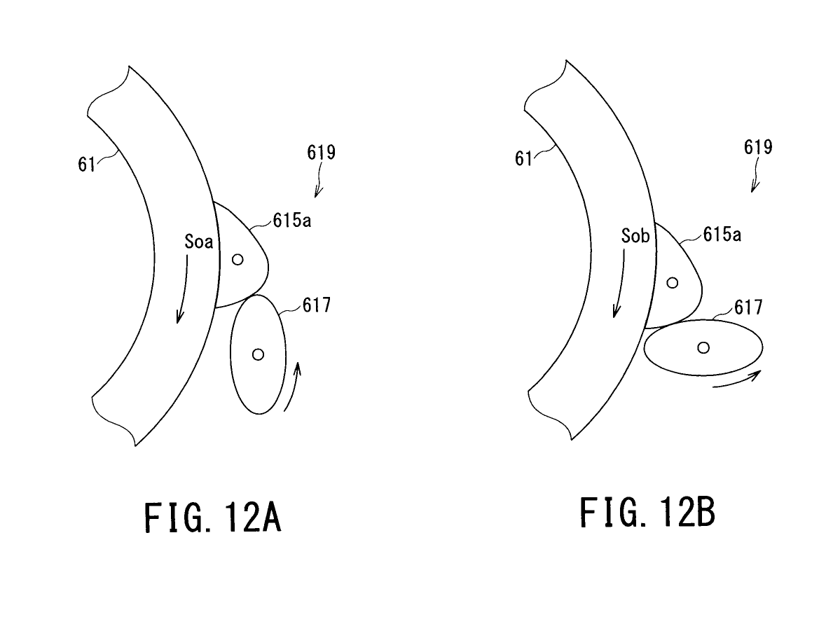

Next, modified versions of the present embodiment will be explained with reference to FIG. 11 and FIG. 12. Although an example in which the reciprocal slide motion of the C-arm 61 in the stereoscopic vision imaging mode is rapidly performed by using the link structure 610 has been explained in the above-mentioned embodiment, this is only an instance. In this modified version, a case where the reciprocal slide motion of the C-arm 61 is rapidly performed by using a cam structure will be explained.

As shown in FIG. 11, the slide moving structure 601 of this modified version includes the belt 611, the rotator 612, the pulleys 613a and 613b, and a cam structure 619.

The belt 611 is mounted along the lateral surface of the C-arm 61. The rotator 612 slides the C-arm 61 in a predetermined direction by moving the belt 611. The pulley 613a changes the running direction of the belt 611 from the lateral surface of the C-arm 61 into the lateral surface of the rotator 612. The pulley 613b changes the above running direction from the lateral surface of the rotator 612 into the lateral surface of the C-arm 61. The cam structure 619 includes a mounting fixture 615a detachable to the lateral surface of the C-arm 61 by a locking means such as an electromagnet etc. and a cam 617 as a rotator whose cross-section is in the form of ellipse, and which rapidly rotates at predetermined rate. The cam 617 and the mounting fixture 615a are in contact with each other by, for example, gravity force, magnetic force, etc. The rotational center of the cam 617 is fixed to a predetermined position. The mounting fixture 615a slides along the outer periphery of the cam 617 in accordance with rotation of the cam 617.

Then, when the standard imaging mode is selected in the operation console 11, the supporting body driving function 81 of the driving, circuit 8 supplies the above-mentioned first driving signal to the slide moving structure 601 of the supporting device 6, and thereby releases the locked state between the mounting fixture 615a of the cam structure 619 and the lateral surface of the C-arm 61 as shown in FIG. 11A.

Next, the supporting body driving function 81 of the driving circuit 8 circulates the belt 611 mounted on the lateral surface of the C-arm 61 under the state where the belt 611 is tensed, by outputting the first driving signal so as to move the rotator 612 to the right. Then, the slide moving structure 601 moves the imaging system mounted near both end parts of the C-arm 61 to the reference imaging position suitable for the standard imaging mode, by sliding the C-arm 61 along its running direction with the use of the belt 611 in accordance with the first driving signal.

Meanwhile, when the stereoscopic vision imaging mode is selected, the supporting body driving function 81 of the driving circuit 8 inputs the first driving signal generated in the same way as the standard imaging mode into the slide moving structure 601, and thereby releases the linked state between the C-arm 61 and the belt 611 mounted on the lateral surface of the C-arm 61 by moving the rotator 612 to the left as shown in FIG. 11B.

Next, the slide moving structure 601 mounts the mounting fixture 615a of the cam structure 619 on the lateral surface of the C-arm 61 and then performs the reciprocal motion of the C-arm 61 within the predetermined angle range Δθ by rapidly rotating the cam 617, in accordance with the first driving signal. Thereby, the slide moving structure 601 makes the imaging system mounted near both end parts of the C-arm 61 rapidly reciprocate between the first and second imaging positions suitable for the stereoscopic vision imaging mode.

FIG. 12A and FIG. 12B show the state where the mounting fixture 615a mounted on the lateral surface of the C-arm 61 rapidly reciprocates in conjunction with rapid rotation of the cam 617 in the stereoscopic vision imaging mode. For example, when the major axis of the cam 617 rapidly rotating counterclockwise is approximately vertically positioned, the central part of the mounting fixture 615a moves to the position Soa corresponding to the first imaging position along the running direction of the C-arm 61. When the major axis of the cam 617 is approximately horizontally positioned, the central part of the mounting fixture 615a moves to the position Sob corresponding to the second imaging position.

In other words, the mounting fixture 615a of the cam structure 619 mounted on the lateral surface of the C-arm 61 repeats the rapid reciprocal motion between the position Soa and the position Sob in conjunction with the rapid rotation of the cam 617, and the C-arm 61 rapidly performs the slide motion in a reciprocating manner along its running direction by the rapid reciprocal motion of the mounting fixture 615a. Then, when the mounting fixture 615a mounted on the C-arm 61 reaches the position Soa, the imaging system mounted near both end pasts of the C-arm 61 is located at the first imaging position suitable for the stereoscopic vision imaging mode. In addition, when the mounting fixture 615a reaches the position Sob, the above-mentioned imaging system is located at the second imaging position.

According to the embodiment and its modified versions of the present disclosure as mentioned above, when the X-ray fluoroscopic imaging of the stereoscopic vision imaging mode is performed, image data compatible with binocular stereoscopic vision having high time resolution can be acquired by rapidly reciprocating the imaging system used for this X-ray fluoroscopic imaging within a predetermined range.

Especially, switching between imaging positions necessary for X-ray fluoroscopic imaging of the stereoscopic vision imaging mode can be repeated in a short time, by rapidly reciprocating the above-mentioned imaging system mounted on a supporting body such as a C-arm etc. with the use of a moving structure having a function of converting rapid rotational motion into rapid reciprocal motion.

In addition, vibration and displacement of the imaging system caused by a moving structure can be greatly reduced by using the above-mentioned moving structure that converts rapid rotational motion into rapid reciprocal motion. Therefore, accurate setting of imaging positions is enabled and high-quality image data compatible with binocular stereoscopic vision can be continuously obtained. Moreover, stable rapid reciprocal motion can be realized for a long term, by using a moving structure whose vibration is small.

In addition, by selectively using a moving structure corresponding to the standard imaging mode and another moving structure corresponding to the stereoscopic vision imaging mode based on selection information on the imaging mode, satisfactory image data can be acquired in the respective imaging modes. Especially, when imaging positions of the stereoscopic vision imaging mode are set on the basis of image data of the standard imaging mode, precise imaging positions can be easily set in a short time. Therefore, examination efficiency and diagnostic accuracy are improved, and burden on an operator is reduced.

Although the embodiment and its modified versions of the present disclosure have been explained above, embodiments of the present disclosure are not limited to the above-mentioned embodiment and its modified versions but can be implemented in a further modified manner.

For example, though an example in which the C-arm 61 is made to rapidly slide by driving the slide moving structure 601 of the supporting device 6 has been explained in above-mentioned embodiment and its modified versions, this is only an instance. For example, imaging positions suitable for binocular stereoscopic vision can be set by driving the holder rotating structure 602 so as to rotate the C-arm 61 fixed to the arm holder 62 around the arm main rotational axis z3 in the b-direction.

In addition, in the case of alternately repeating (a) X-ray fluoroscopic imaging of the stereoscopic vision imaging mode at the first imaging position and (b) X-ray fluoroscopic imaging of the stereoscopic vision imaging mode at the second imaging position, an example in which display data compatible with binocular stereoscopic vision are generated at the timing of completing generation and storage of the second image data in the second imaging position by using (A) the first image data having been already acquired and stored in the image data memory circuit 92a and (B) the second image data stored in the image data memory circuit 92b has been explained in the above-mentioned embodiment.

However, this is only an example. The X-ray imaging apparatus 100 may be configured so that (a) new display data are generated by using newly acquired first image data and the second image data having been already acquired and stored in image data memory circuit 92b when the first image data are newly acquired, and (b) new display data are generated by using newly acquired second image data and the first image data having been already acquired and stored in image data memory circuit 92a when the second image data are newly acquired following the above new first image data.

Meanwhile, an example in which the imaging positions of the stereoscopic vision imaging mode are alternately switched by rapidly reciprocating the imaging system mounted near both end parts of the C-arm 61 within a predetermined range has been explained in the above embodiment and its modified versions. However, this is only an example. For instance, imaging positions suitable for binocular stereoscopic vision may be set by rapidly reciprocating an imaging system mounted on anther supporting body such as an Q arm etc.

Incidentally, the respective components included in the X-ray diagnostic apparatus 100 of the present embodiment and its modified versions can be achieved by, for example, using a computer composed of a CPU, a RAM, a magnetic memory device, an input device, a display device, etc. as hardware.

The above-mentioned processor means, for instance, a circuit such as a dedicated or general-purpose CPU (Central Processing Unit), a dedicated or general-purpose GPU (Graphics Processing Unit), an ASIC (Application Specific Integrated Circuit), a programmable logic device including SPLD (Simple Programmable Logic Device) and CPLD (Complex Programmable Logic Device) as examples, and an FPGA (Field Programmable Gate Array), and so on. A processor achieves various types of functions by reading out programs stored in a memory circuit and implementing the programs.

In addition, programs may be directly installed in the circuit of a processor instead of storing programs in the memory circuit. In this case, the processor achieves various types of functions by reading out programs stored in its own circuit and implementing the programs.

In addition, the system control circuit 12, the driving circuit 8, and the image data generation/memory circuit 9 may be configured as mutually separate processors (i.e. first processing circuitry, second processing circuitry, and third processing circuitry), or these three circuits may be configured as unified processing circuitry. In addition, when a plurality of processors is used, a memory circuit (or a memory medium) for storing programs may be disposed for each processor.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An X-ray diagnostic apparatus comprising:
an X-ray generator configured to generate X-rays to be irradiated on an object;
an X-ray detector configured to detect the X-rays;
a supporting body configured to support the X-ray generator and the X-ray detector; and
a supporting body moving structure configured to be provided on the supporting body, to include a rotator, and to reciprocate the supporting body by rotational motion of the rotator in one direction.

2. The X-ray diagnostic apparatus according to claim 1, wherein
the supporting body moving structure is a cam structure or a link structure.

3. The X-ray diagnostic apparatus according to claim 1, further comprising:
an image generation circuit configured to generate an X-ray image based on the X-rays detected by the X-ray generator; and
a display configured to display the X-ray image generated during the rotational motion.

4. The X-ray diagnostic apparatus according to claim 3, wherein
the image generation circuit is configured to generate a plurality of X-ray images during the rotational motion, and
the display is configured to display a stereoscopically visible image based on the plurality of X-ray images.

5. The X-ray diagnostic apparatus according to claim 1, further comprising an operation console configured to receive an instruction input for switching between a reciprocal motion mode and a normal mode, in the reciprocal motion mode the supporting body reciprocating within a predetermined range along a predetermined direction, and in the normal mode the supporting body being positioned at a predetermined position by moved along the predetermined direction.

6. The X-ray diagnostic apparatus according to claim 5, wherein
the X-ray generator repeatedly performs irradiation of X-rays at least at two predetermined positions while the supporting body is reciprocating in the reciprocal motion mode.

7. The X-ray diagnostic apparatus according to claim 6, further comprising:
an image generation circuit configured to generate a left eye image based on X-rays detected at one of the two predetermined positions, and to generate a right eye image based on X-rays detected at another of the two predetermined positions; and
a display configured to display a stereoscopically visible image based on the left eye image and the right eye image.

8. The X-ray diagnostic apparatus according to claim 6, wherein
the two predetermined positions are turnaround points of a movement direction of reciprocal motion of the supporting body.

9. The X-ray diagnostic apparatus according to claim 6, wherein
the supporting body moving structure is configured to further include a belt connected with the supporting body, to reciprocate the supporting body within the predetermined range along the predetermined direction by rotational motion of the rotator in one direction in the reciprocal motion mode, and to move the supporting body along the predetermined direction according to a movement of the belt in the normal mode so as to position the supporting body at the predetermined position.
